(19) United States
(12) Patent Application Publication
(10) Pub. No.: US 2007/0005061 A1
(43) Pub. Date: Jan. 4, 2007

(54) Transvaginal uterine artery occlusion

(75) Inventors: Joseph Eder, Los Altos Hills, CA (US); Camran Nezhat, Woodside, CA (US); John Maroney, Woodside, CA (US); Roger A. Stern, Cupertino, CA (US)

Correspondence Address: GLENN PATENT GROUP, 3475 EDISON WAY, SUITE L, MENLO PARK, CA, 94025, US

(73) Assignee: Forcept, Inc., Menlo Park, CA (US)

(21) Appl. No.: 11/173,478

(22) Filed: Jun. 30, 2005

(51) Int. Cl. A61B 18/14

(52) U.S. Cl. 606/51; 606/158

(57) ABSTRACT

Uterine artery occlusion is performed for the treatment of uterine fibroid using a tool which is introduced through the vaginal wall to the exterior of the uterus. The tool carried clamping elements which may be positioned over the uterine artery. Electrodes or other energy applying devices on the clamping elements may be used to deliver energy to seal the uterine artery. Optionally, the tool may carry ultrasonic, visual, or proximity sensors for detecting the presence of the uterine artery prior to delivering energy.

### FIELD OF THE INVENTION

The present invention relates generally to medical devices and methods. More particularly, the present invention relates to minimally invasive methods and apparatus for performing uterine artery occlusion for the treatment of fibroids.

Uterine fibroids, also referred to as uterine myomas, affect a large number of women, although most fibroids are symptom free and do not require treatment. Fibroids, however, can be problematic if they grow rapidly, are large enough to displace other organs, such as the bladder, cause fertility problems, or lead to abnormal bleeding.

A number of therapies are available for treating uterine fibroids, including myomectomy, laparoscopic myomectomy, hysterectomy, fibroid embolization, and uterine artery embolization. Of particular interest to the present invention, uterine artery embolization relies on blocking or occluding the arteries that supply blood to the fibroids. A catheter is introduced to the uterine arteries under fluoroscopy, and small particles are injected into the arteries in order to block blood flow. Blocking the blood supply can shrink the fibroids in order to reduce or eliminate symptoms.

Although promising, intravascular embolization can be undesirable for a number of reasons, including ineffectiveness and patient incompatibility. Recently, it has been proposed to occlude the uterine artery in other ways, such as, using a radiofrequency ablation needle introduced through the uterine wall, optionally under the transrectal or other imaging. U.S. Patent No. 6,905,506, describes a transvaginal approach for clamping the cervix to temporarily occlude the uterine artery and allow the fibroid to shrink. None of these approaches, however, is wholly effective or suitable for all patients. Thus, there remains a need for providing alternative methodologies, protocols, and apparatus for performing fibroid treatment by occlusion of the uterine arteries.

### DESCRIPTION OF THE BACKGROUND ART

U.S. Pat. No. 6,905,506 describes a method for reversibly compressing the uterine arteries using a clamp introduced to the cervix through the vagina. Clamping devices with radiofrequency electrodes are described in U.S. Pat. Nos. 6,059,782 and 5,746,750. U.S. Pat. No. 6,059,766 devices a method of embolotherapy which introduces embolic elements into uterine arteries through the uterine wall. The following U.S. Patents may also be relevant to the present invention: U.S. Pat. Nos. 3,920,021; 3,845,771; 4,041,952; 4,671,274; 4,972,846; 5,037,379; 5,078,736; 5,151,102; 5,178,618; 5,207,691; 5,217,030; 5,267,998; 5,269,780; 5,269,782; 5,281,216; 5,282,799; 5,290,287; 5,295,990; 5,300,087; 5,324,289; 5,330,471; 5,336,229; 5,336,237; 5,342,381; 5,352,223; 5,352,235; 5,356,408; 5,391,166; 5,395,369; 5,396,900; 5,403,312; 5,417,687; 5,423,814; 5,445,638; 5,456,684; 5,458,598; 5,462,546; 5,482,054; 5,484,435; 5,484,436; 5,496,312; 5,496,317; 5,514,134; 5,531,744; 5,540,684; 5,540,685; 5,542,945; 5,549,606; 5,558,100; 5,558,671; 5,569,243; 5,573,535; 5,578,052; 5,599,350; 5,603,711; 5,611,803; 5,624,452; 5,637,110; 5,637,111; 5,653,692; 5,658,281; 5,665,085; 5,665,100; 5,667,526; 5,669,907; 5,674,184; 5,674,220; 5,681,282; 5,683,385; 5,683,388; 5,688,270; 5,693,051; 5,697,949; 5,700,261; 5,702,390; 5,707,369; 5,709,680; 5,713,896; 5,718,703; 5,733,283; 5,735,289; 5,735,848; 5,735,849; 5,741,285; 5,743,906; 5,755,717; 5,833,690; 6,602,251; 6,743,229, 6,746,488; and US2001/0014805.

### BRIEF SUMMARY OF THE INVENTION

The present invention provides improved methods, apparatus, and systems for performing uterine artery occlusion for the treatment for uterine fibroids. According to the methods of the present invention, a tool is advanced through a vaginal wall to the uterine artery (or other artery feeding the uterus), and the tool is used to compress and apply energy to occlude the artery. The tool is preferably introduced transvaginally to a location on the vaginal wall adjacent to the cervix, typically at or near a fornix of the vagina. The vaginal wall will be penetrated, typically by making one, two, or several small incisions under direct visualization using conventional, surgical instruments. Alternatively, the tool which is introduced may itself have penetrating element, such as a blade, electrosurgical tip, or the like, in order to introduce the tool directly through the vaginal wall without a prior incision.

After the compressing tool has been introduced through the vaginal wall, it will be advanced toward the uterine or other target artery. Preferably, before the artery is compressed and/or energy is applied, the position of the tool adjacent to the uterine artery will be confirmed. Optionally, a visual or audible signal will be given when the tool is properly positioned. Confirming may comprise visualizing the tool and/or the uterine artery in any one of several ways. For example, the location of the tool relative to the uterine artery can be confirmed using laparoscopic imaging according to conventional gynecological procedures. Alternatively, the position to the tool relative to the uterine artery may be determined using external ultrasound, fluoroscopic, or other imaging. Alternatively or in addition to either laparoscopic, ultrasonic or fluoroscopic imaging, the imaging tool may carry its own optical or ultrasound imaging element in order to confirm positioning. In any event, after the device has been properly positioned, it is used to compress and apply energy to the uterine or other target artery to achieve occlusion.

In still further embodiments, the devices of the present invention may rely on blood flow detection to confirm proximity of the target artery. In such embodiments, a Doppler ultrasound element will be positioned at or near the distal end of the tool, and presence of the artery can be detected by conventional ultrasound detection and methods. Other techniques for confirming position include proximity sensing, pressure sensing, and the like.

In the exemplary embodiments, the tool comprises opposed clamping elements which effect clamping of the uterine artery. The clamping elements will typically carry electrodes or other energy (or cryotherapy) delivering components to permit permanent occlusion of the artery while it is being temporarily clamped by the clamping elements. The energy will be applied under conditions which seal the artery lumen but which leave the artery otherwise intact to avoid the need for hemostasis. The preferred energy to be delivered is radiofrequency (RF), but other energy including heat energy, ultrasonic energy, microwave energy, mechanical energy, and the like, might also be suitable. Alternatively, the tool may carry one or more fasteners, such as clips, staples, suture loops, or the like, which can be mechanically deployed to constrict the vessel.

The present invention still further provides devices for occluding the uterine or other target artery via a transvaginal approach. Such devices comprise a shaft structure having opposed clamping elements near its distal end. The shaft structure will adapted to be positioned through a vaginal wall (preferably from the vaginal cavity) to position the distal end thereof adjacent to the uterine artery. The clamping elements will have electrodes or other structures for applying energy to the uterine artery when the uterine artery is clamped therebetween. Preferred energy delivering structures are radiofrequency electrodes, but other structures would be suitable as well.

In a first exemplary embodiment, the shaft comprises a pair of hinged arms each of which carry at least one electrode, preferably a radiofrequency electrode connectable to a monopolar or bipolar power supply. In a preferred embodiment, at least one of the arms will also carry an imaging or a Doppler ultrasound element in order to permit confirmation that the clamps are adjacent to the uterine artery.

In an alternate embodiment, the shaft may consist essentially of a singular tubular element having an advanceable clamping element therein. The use of a single tubular element can be advantageous as it is easier to introduce through a small incision in the vaginal wall and does not require opening and closing of arms as with the hinged embodiments.

A variety of other clamping mechanisms would also be available, including parallelogram linkages, bimetallic actuators, solenoid devices, motorized operators, and the like.

The present invention still further provides systems for occluding uterine arteries, where the systems comprise any of the devices described above in combination with a power supply and control unit for applying energy through the energy applying means on the device. The power supply will typically be configured to delivery radiofrequency energy, but any of the other energy sources described above would also be suitable. The system will still further comprise a Doppler or optical imaging or sensing systems for confirming the presence of the device adjacent to the uterine artery prior to treatment.

### BRIEF DESCRIPTION OF THE DRAWINGS

FIG. 1 illustrates the right and left uterine arteries in position relative to a patient's vagina and uterus.

FIG. 2 illustrates a first exemplary treatment tool constructed in accordance with the principles of the present invention.

FIGS. 3A and 3B illustrate alternative constructions of a distal end of the tool of FIG. 2, taken along line 3-3.

FIGS. 4A and 4B illustrate an alternative embodiment of the treatment tool of the present invention.

FIGS. 5A-5E illustrate the tool of FIG. 2 being used for uterine artery occlusion in accordance with the principles of the present invention.

### DETAILED DESCRIPTION OF THE INVENTION

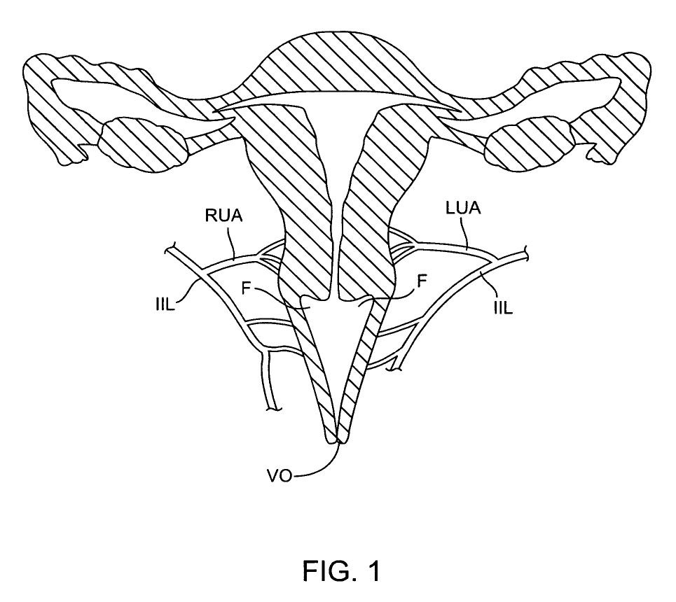

According to FIG. 1, a patient's right uterine artery RUA and left uterine artery LUA branch from the right and left internal iliac arteries (IIL) and enter into the walls of the uterus along a medial plain. The present invention provides for accessing the uterine arteries or other target arteries by placing a tool through the vagina V, advancing the tool upward through the vagina to a fornix F adjacent to the cervix C.

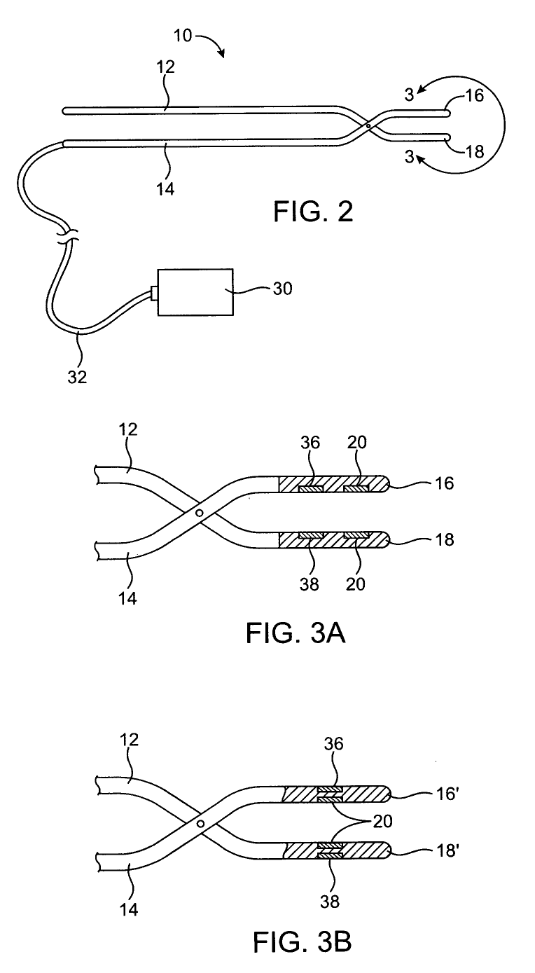

A variety of tools can be used for accessing and penetrating through the uterine wall in the region of the fornix F to access the uterine artery UA. Referring to FIGS. 2, 3A, and 3B, a first device 10 comprises a pair of hinged arms 12 and 14 having distal clamping elements 16 and 18, as best illustrated in FIG. 2. The distal clamping elements 16 and 18 will carry a mechanism or structure for delivering energy (or cold) to the uterine artery when the uterine artery is clamped therebetween. The exemplary embodiments, the mechanism will comprise a pair opposed electrodes 20 suitable for delivering radiofrequency energy which may delivered from a power supply and control unit 30 which is connected to the device 10 via a cable 32 (FIG. 2).

Preferably, the clamping elements 16 and 18 will also comprise a mechanism or structure for confirming proximity of the uterine artery UA. As illustrated in FIG. 3A, a pair of ultrasonic transducers 36 and 38 are mounted proximally of the electrodes 20. The ultrasonic transducers preferably configured for Doppler ultrasound sensing of blood flow through the uterine artery UA, allowing generation of a simple visual or audible signal to confirm proper placement of the device. Alternatively, the ultrasonic elements could provide for ultrasonic imaging in a conventional manner, or could in some cases comprises optical imaging, components, such as optical fibers, CCD's or the like. Still further alternatively, presence of the uterine artery can be sensed with a proximity sensor, pressure sensor, or other device which can provide visual or audible feedback when the clamping elements 36 and 38 are adjacent to the uterine artery UA.

As an alternative to the distal end of FIG. 3A, FIG. 3B describes clamping arms 16′ and 18′ where the electrodes 20 and ultrasonic transducers 36 and 38 are stacked above each other rather than positioned adjacent to each other in the axial direction.

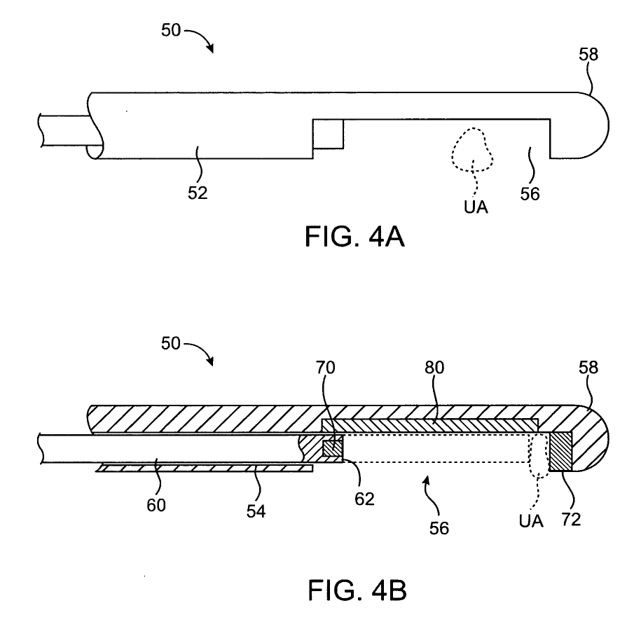

A number of other specific devices can be configured for performing the methods of the present invention. For example, as illustrated in FIGS. 4A and 4B, a treatment device 50 may comprise a single shaft 52 performed as a tube having at least one lumen 54 therein. A gap 56 is provided near a distal end 58 of the shaft, and a sliding clamping element 60 can pass through the lumen 54 and have a distal end 62 and/or an advance through the gap 56. As shown in FIG. 4B, the distal end 62 of the element 60 may comprise an electrode 70 or other energy delivering component. Similarly, an electrode 72 or other energy delivering component may be disposed in a distal surface of the gap within the shaft 52. Preferably, an ultrasonic or other position sensor 80 could be provided along an axial wall of the gap 56 in order to permit detection of the uterine artery UA when the uterine artery is in the gap 56. Clamping of the uterine artery can be achieved by advancing the clamping element 60 in a distal direction, as shown in broken line in FIG. 4B, to collapse the uterine artery between the electrodes 70 and 72. Radiofrequency or other energy may then be delivered into the uterine artery in order to fuse the lumen and induce occlusion of the lumen of the uterine artery.

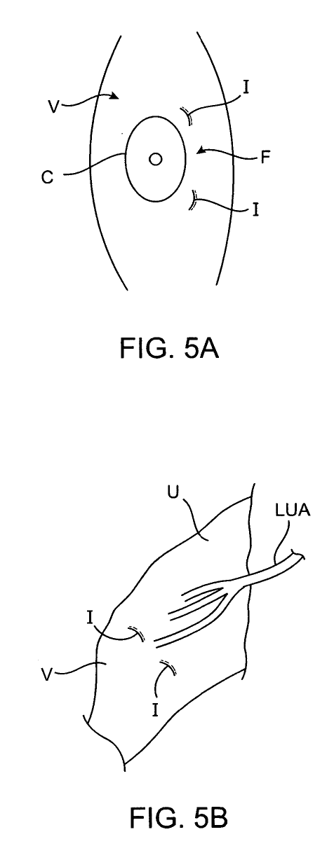

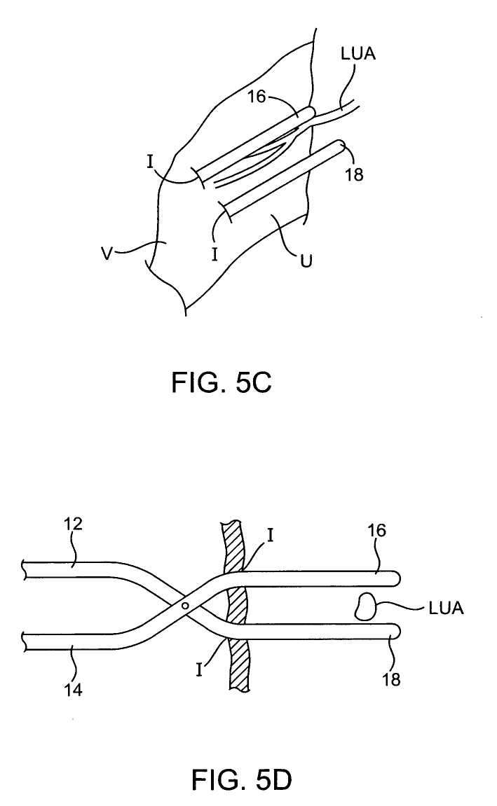

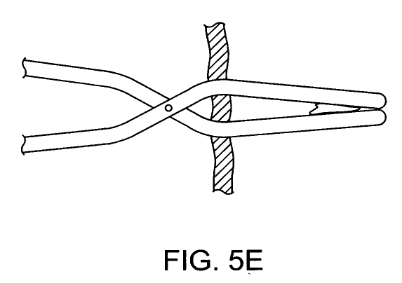

Referring now to FIGS. 5A though 5E use of the device 10 for occluding a uterine artery UA in accordance with the principles to the present invention will be described. Initially, the treating physician visualizes the cervix C through the vagina V using conventional tools and techniques, as illustrated in FIG. 5A. One or more small incisions I may be made in the region of a fornix F of the rear vaginal wall. The incisions I will extend to the exterior of the vagina V at the base of the uterus U, as best seen in FIG. 5B the incisions I will be relatively close to the left uterine artery LUA.

Clamping elements 16 and 18 will be advanced through the Incisions so that they lie on the anterior and posterior sides of the left uterine artery LUA, as best seen in FIG. 5C. An alternate view is also shown in FIG. 5D. The arms 12 and 14 are then manipulated to collapse the clamping elements 16 and 18 over the uterine artery LUA as shown in FIG. 5E. Usually, prior to clamping, correct positioning of the clamping element 16 and 18 will be confirmed via the Doppler or other ultrasonic elements carried by the device. Assuming correct positioning, the uterine artery is clamped, and energy applied in order to permanently fuse and occlude the lumen of the uterine artery, as shown in FIG. 5E. Although the type and amount of energy may vary widely, radiofrequency energy at a power from 5 W to 300 W, typically from 10 W to 50 W, from 1 second to 30 seconds, should be sufficient to achieve permanent occlusion.

After the occlusion has been performed, for devices carrying the Doppler ultrasound, it will be possible to confirm that blood flow through the artery has ceased prior to withdrawing the device through the incisions I and vaginal opening. The incisions I may then be closed, and the procedure has ended.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for treating uterine fibroids, said method comprising:
advancing a tool through a vaginal wall to an artery which feeds the uterus, using the tool to compress and apply energy to occlude the artery.

2. A method as in claim 1, wherein the tool is advanced through a location in the vaginal wall adjacent to the cervix.

3. A method as in claim 1, further comprising penetrating the vaginal wall with a tool.

4. A method as in claim 3, wherein the tool which penetrates the vaginal wall is a different tool than the one which compresses and applies energy to the artery.

5. A method as in claim 3, wherein the tool which penetrates the vaginal wall is the same tool as the one which compresses and applies energy to the artery.

6. A method in which claim 1, further comprising confirming that the tool is adjacent to the artery prior to using the tool to compress and apply energy to occlude the artery.

7. A method as in claim 6, wherein confirming comprises visualizing the tool and/or the artery.

8. A method as in claim 7, wherein visualizing comprises laparoscopic imaging of the artery.

9. A method as in claim 7, wherein visualizing comprises external imaging using ultrasound or fluoroscopy.

10. A method as in claim 7, wherein visualizing comprises rectal imaging using ultrasound.

11. A method as in claim 7, wherein visualizing is performed using an imaging element on the tool which is used to compress and apply energy to the artery.

12. A method as in claim 6, wherein confirming comprising detecting, proximity of the tool to blood flow through the artery.

13. A method as in claim 6, wherein detecting is performed using a Doppler ultrasound element on the tool.

14. A method as in claim 1, wherein using the tool comprises clamping opposed clamping elements of the tool on the artery and applying energy through the clamping elements to the artery under conditions which seal the artery lumen but leave the artery otherwise intact.

15. A method as in claim 1, wherein the tool delivers radiofrequency, energy to the artery.

16. A device for occluding an artery which feeds the uterus, said device comprising:
a shaft structure adapted to be positioned through a vaginal wall to position a distal end thereof adjacent to the artery;
opposed clamping elements on the shaft near the distal end; and
means for applying energy from the clamping elements to the artery when the artery is clamped therebetween.

17. A device as in claim 16, wherein the shaft comprises two hinged arms each of which carries at least one electrode.

18. A device as in claim 17, wherein at least one arm carries a proximity sensor.

19. A device as in claim 18, wherein the proximity sensor comprises a Doppler ultrasound element.

20. A device as in claim 16, wherein the shaft consists essentially of a single tubular element having an advanceable clamping element therein.

21. A device as in claim 20, wherein the tubular elements and the advanceable clamping element carry opposable electrodes.

22. A device as in claim 21, wherein the shaft further carries a proximity sensor.

23. A system for occluding an artery which feeds the uterus, said system comprising:
a device as in any one of claims 16 to 22;
a power supply and control unit for applying energy through the energy applying means to the artery.

24. A system as in claim 23, wherein the power supply delivers radiofrequency energy to the energy applying means.

25. A system as in claim 24, wherein the power supply and control unit further comprises a proximity sensor which receives signals from the device when the distal end is adjacent to the artery.

26. A system as in claim 24, further comprising an audible or visual signal when the energy applying means is positioned adjacent to the artery.
